(19) United States
(12) Patent Application Publication
(10) Pub. No.: US 2021/0212306 A1
(43) Pub. Date: Jul. 15, 2021

(54) SANITIZING AND INSECT-TRAPPING DEVICE

(71) Applicant: U WELL TECH CORP., Apia (SM)

(72) Inventors: JAU-CHEN TSAI, TAIPEI CITY (TW); HAO-CHUAN FU, NEW TAIPEI CITY (TW); CHAO-CHI TSAI, TAIPEI CITY (TW); KUAN-HAO FU, NEW TAIPEI CITY (TW)

(21) Appl. No.: 17/213,781

(22) Filed: Mar. 26, 2021

Related U.S. Application Data
(63) Continuation-in-part of application No. 16/242,156, filed on Jan. 8, 2019.

(51) Int. Cl. A01M 1/14; A01M 1/04; A01M 1/02; A61L 2/08; A61L 2/10

(52) U.S. Cl. CPC A01M 1/145; A01M 1/04; A01M 1/026; A61L 2202/14; A61L 2/10; A61L 2202/11; A61L 2/088

(57) ABSTRACT

A sanitizing and insect-trapping device includes a casing, at least one LED light bar, a board, and titanium dioxide photocatalyst. The casing includes a plurality of openings. The at least one LED light bar is disposed of inside the casing and has LEDs disposed inside a metal hood and arranged to emit ultraviolet light. The board includes a light diffusion agent and is arranged inside the casing to be exposed to the ultraviolet light emitting from the LEDs. The titanium dioxide photocatalyst is arranged inside the casing to be exposed to ultraviolet light. The device provides functions of sterilization, insect trapping, air purification and also shows advantages of extended usage time life and low harm to human health.

### CROSS-REFERENCE TO RELATED APPLICATIONS

This application is a continuation-in-part of U.S. patent application Ser. No. 16/242,156, filed on Jan. 8, 2019 and entitled INSECT TRAP DEVICE, the complete subject matter of which is incorporated herein as reference.

### FIELD OF THE INVENTION

The present invention relates to a device having functions of sterilization and insect trapping, and more particular to a device that involves improvement on the installation of an ultraviolet light bar, which helps prevent light from directly irradiating human bodies, and also functions to enhance an insect trapping effect and for sterilization and air purification, as being used in combination with titanium dioxide photocatalyst.

### BACKGROUND OF THE INVENTION

Ultraviolet light has the functions of sanitization and disinfection. Many households have ultraviolet lights for sterilization, mothproofing, air purification, and allergen elimination, in order to improve living quality. However, the ultraviolet sterilization lights that are available in the market often expose a lighting tube to the outside, causing the high intensity of high ultraviolet light around the light source, which when contacting human bodies would cause damages to the eyes and skins of the human bodies for exposure in long-term of time.

Further, ultraviolet light exhibits the property of attracting insects to approach, and is thus often employed for insect trap lanterns. Insect trap lanterns are often set up in agricultural sites, such as vegetable gardens, fruit farms, barns, and vegetable cultivation greenhouses, or are provided in for example restaurants, hotels, supermarkets, factories, hospitals, schools, parking lots, and living environments, to catch and trap harmful insects that cause damage to growth and preservation of corps and causes problems to human living.

However, the known insect trap lanterns are structure to have an ultraviolet light source directly exposed, and are thus dangerous to human health. Further, after insects are trapped, there is no measure to destroy bacteria carried by the insects. Further, circuit boards, wires, and fasteners that are included in the known insect trap lantern are of ten long irradiated with the ultraviolet light source, and this leads to the reduction of usage time life. Consequently, there are further improvements needed.

### SUMMARY OF THE INVENTION

In view of the above problems, the primary objective of the present invention is to provide a sanitizing and insect-trapping device having functions of sterilization and insect trapping, which possesses the functions of air purification, sterilization, and deodorization and also helps take care of human health and extends the usage time life, and, if desired, may also serve as an insect trap lantern.

To achieve the above objective, the present invention provides a sanitizing and insect-trapping device, which comprises a casing, the casing has formed with a plurality of openings; at least one LED light bar, the at least one LED light bar being arranged in an interior of the casing, each of the at least one LED light bar comprising a metal hood and a plurality of LEDs, the metal hood comprising a plurality of openings, wherein the LEDs is disposed inside the metal hood at a location corresponding to the openings of the metal hood and is arranged to emit ultraviolet light; a board, which comprises a light diffusion agent, the board is arranged in the interior of the casing and being exposed to the ultraviolet light emitting from the LEDs; and titanium dioxide photocatalyst, which is arranged in the interior of the casing, wherein the titanium dioxide photocatalyst is disposed on metal and is exposed to the ultraviolet light emitting from the LEDs.

In an embodiment, the LEDs have a height that is lower than the height of the openings of the metal hood.

In an embodiment, a fan is further included and is disposed in the casing, and the fan is arranged to draw external air into the casing and to blow air and gases inside the casing to the outside of the casing so as to circulate air.

In an embodiment, the LEDs are arranged to emit long-wavelength ultraviolet light, the long-wavelength ultraviolet light having a wavelength in the range of 350-380 nm.

In an embodiment, the wavelength of the long-wavelength ultraviolet light is in the range of 355-370 nm.

In an embodiment, the casing is further provided, in the interior thereof, with a monitoring module and a metal barrier plate, wherein the metal barrier plate defines, together with a wall of the casing, an installation space, and a circuit board and wirings of the monitoring module are been install in the installation space.

In an embodiment, the monitoring module comprises a control unit, an image-capturing unit, a humidity and temperature sensor, an infrared sensor, a transmission unit, an ultrasonic mouse repelling unit, and a photosensor, and the image-capturing unit, the humidity and temperature sensor, the infrared sensor, the transmission unit, the ultrasonic mouse repelling unit, and the photosensor are each in electrical connection with the control unit.

In an embodiment, the monitoring module further comprises a display unit, and the display unit is disposed of on the casing and in electrical connection with the control unit.

In an embodiment, the control unit is in communication connection with a temperature regulation device and a humidity regulation device by means of the transmission unit.

In an embodiment, the control unit is in communication connection with a computation device by means of the transmission unit.

In an embodiment, the casing is provided with an electrical power unit to supply electrical power.

In an embodiment, the electrical power unit comprises one of a direct-current power supply, an alternate-current power supply, a battery, and a solar cell, or a combination thereof.

In an embodiment, the casing is provided with a wall-mounting aperture for mounting to a wall.

In an embodiment, the board has a surface that is coated with insect sticking glue.

In an embodiment, the board has a surface that is formed with grid lines.

In an embodiment, the casing is made of a anti-ultraviolet material and at least one LED light bar is fixed in the interior of the casing by means of at least one metal clip.

The sanitizing and insect-trapping device according to the present invention uses ultraviolet light emitting from LEDs to attract insects to approach and the ultraviolet light irradiates titanium dioxide photocatalyst to generate hydroxide ions that help break down bacteria, germs, and bacteria carried by insect bodies to generate carbon dioxide in order to achieve the effects of sterilization and deodorization and is thus beneficial to human health.

The sanitizing and insect-trapping device according to the present invention may selectively serve as an insect trap device, which can be used in a certain period of time, season, or a site in which insects actively appear.

Further, the sanitizing and insect-trapping device according to the present invention is provided with a fan to draw in external air and to blow carbon dioxide converted from bacteria and germs carried in air and bacteria carried by caught insects to the outside to attract insects, further improve the efficiency of catching insects. Also, an effect of circulation and purification of air may be achieved.

In addition, the LEDs and a circuit board and wirings thereof are all disposed of in the interior of a metal hood, so that the intensity of ultraviolet light around the sanitizing and insect-trapping device is insignificant and the damage to human bodies as being irradiated with ultraviolet light can be reduced, and damage of the circuit board and wirings by ultraviolet light is also alleviated to extend the usage time life. In some embodiments, the sanitizing and insect-trapping device according to the present invention comprises a monitor unit and the monitor unit is in connection with a computation device, a temperature regulation device, or a humidity regulation device, to instantaneously collect images of insects caught by the sanitizing and insect-trapping device and detect the humidity and temperature of the surrounding environment to allow an operator to do comparison and analysis based on the types and numbers of the caught insects and the humidity and temperature data, and to allow change of the humidity and temperature of the surrounding environment, on-site or remote, to reduce insects of specific types.

Thus, the sanitizing and insect-trapping device according to the present invention possesses functions and advantages that include circulation and purification of air, sterilization, deodorization, protection of human bodies from damage by ultraviolet light sources, extension of usage time life, energy-saving and environmental protection, simplified and compact overall structure, and easy installation. In serving as an insect trap device, the present invention shows an increased insect catch rate as compared to the known insect trap lanterns and provides the convenience of reading the types of insects caught in an instantaneous and remotely-monitoring manner, so as to be helpful for pest control. The sanitizing and insect-trapping device according to the present invention demonstrates multiple uses and involves severe consideration for the health and safety of human bodies.

### BRIEF DESCRIPTION OF THE DRAWINGS

FIG. 1 is a perspective view showing a sanitizing and insect-trapping device according to an embodiment of the present invention;

FIG. 2 is a perspective view showing the sanitizing and insect-trapping device from a different angle;

FIG. 3 is an exploded view showing the sanitizing and insect-trapping device according to the present invention;

FIG. 4 is a cross-sectional view taken along line A-A of FIG. 2;

FIG. 5A is an enlarged view of a circled portion B of FIG. 4, showing a section of a light-emitting diode (LED) light bar;

FIG. 5B is a perspective view of the LED light bar according to the present invention; and

FIG. 6 is a block diagram of a monitoring module of the sanitizing and insect-trapping device according to the present invention.

### DETAILED DESCRIPTION OF THE PREFERRED EMBODIMENTS

In order to demonstrate the concept and effect of the present invention, a description will be provided below with reference to an embodiment and the attached drawings. It is noted that the embodiment described hereinafter in this specification and the drawings are provided to helping understand the present invention, and should not be construed as a limitation to the present invention.

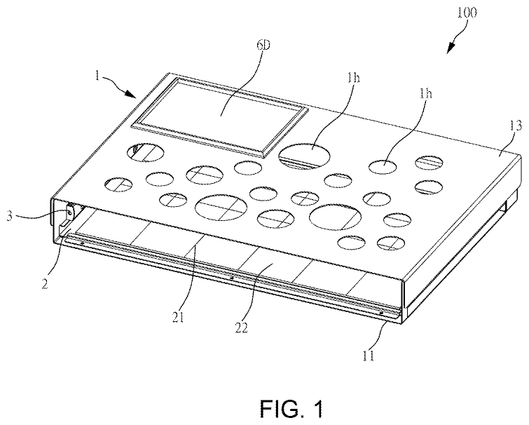

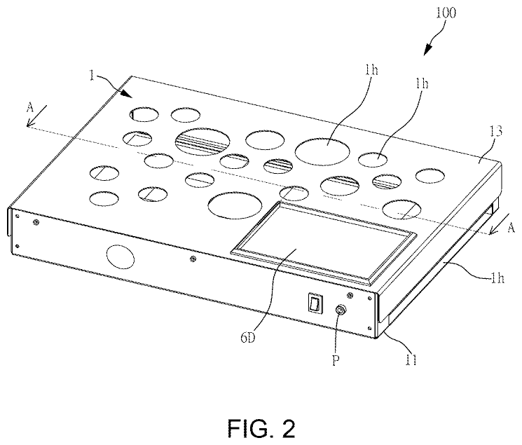

FIGS. 1-6 illustrates an embodiment of a sanitizing and insect-trapping device according to the present invention. The sanitizing and insect-trapping device 100 comprises a casing 1, a board 2, at least one light-emitting diode (LED) light bar 3, and titanium dioxide photocatalyst 4.

As shown in FIGS. 1-3, the casing 1 is formed with a plurality of openings 1h. The openings 1h communicate between inside and outside of the casing 1. Casing 1 can be made up of various parts or integrally formed as one piece. Casing 1 may have a shape that includes a three-dimensional shape of any proper form. In the instant embodiment, casing 1 comprises a top cover 13 and a base 11. The base 11 has two opposite sidewalls 111 that are provided with metal clips 112 for mounting and fixing the LED light bar 3. Casing 1 is provided with a pair of wall-mounting apertures 114 for purpose of being mounted to a wall. In an embodiment, casing 1 is made of a material that is anti-ultraviolet material in order to prevent material deterioration resulting from irradiation of ultraviolet light. Casing 1 can be made of a material that includes aluminum and bamboo.

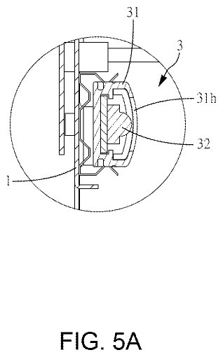

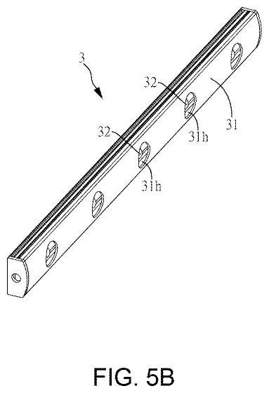

As shown in FIGS. 5A and 5E, which is at least one LED light bar 3 arranged inside the casing 1. Each of the at least one LED light bar 3 comprises a metal hood 31 and a plurality of LEDs 32. The metal hood 31 comprises a plurality of openings 31h. The LEDs 32 are arranged inside the metal hood 31 at a location corresponding to the openings 31h of the metal hood 31 and are arranged to give off ultraviolet light for attracting insects to approach. Compared to ultraviolet light tubes, the LEDs 32 has the advantages of energy-saving and environment conservation as using only a small electrical current and a low voltage. In the embodiment of FIG. 5A, the LEDs 32 are arranged to have a height that is lower than the height of the openings 31h formed in the metal hood 31, and the LEDs 32 are spaced from the ones of the openings 31h corresponding thereto by a distance of 0.1-5 mm. This arrangement sets up a range of ultraviolet light emitting from the LEDs 32, meaning ultraviolet light is transmittable out through the openings 31h.

Since the LEDs 32 are arranged inside the metal hood 31, it is possible to reduce the intensity of ultraviolet light around the sanitizing and insect-trapping device 100 and also to reduce radiation and radiating heat that a human body may contact to thereby prevent damage to human health. The sanitizing and insect-trapping device 100 according to the present invention exhibits only very minor ultraviolet light inspected outside the casing 1, and the specification also meets the standards of IEC 62471 and EN 62471, helping lowering potential hazard of human skin, eyes, and retinas caused by ultraviolet light, blue light, thermal energy, and infrared radiation. Further, as shown in FIG. 5E, a major portion of a circuit board and wirings of the LEDs 32 are shielded by the metal hood 31, so that deterioration caused by irradiation of ultraviolet light can be reduced to thereby extending the life span thereof.

Board 2 comprises a light diffusion agent. Board 2 is arranged inside casing 1 and in contact with ultraviolet light emitting from the LEDs 32. When ultraviolet light irradiates board 2, board 2 helps homogenize the ultraviolet light.

Optionally, in an embodiment, in a season of insects and mosquitos, board 2 has a surface coated with insect sticking glue 22 to trap and catch insects that are attracted by the ultraviolet light to get into the casing 1. Insects, upon being attracted by ultraviolet light to get into casing 1 by passing through the plurality of openings 1h of the casing 1, are readily caught by the board 2 that is coated with the insect sticking glue 22. Thus, if desired, the sanitizing and insect-trapping device 100 may serve as an insect trap device and be used in a certain period of time, or a season, or a site, when or where the insects are active. In an embodiment, board 2 is provided, on a surface thereof, with grid lines 21 to help an operator to make a rough estimation of the types and numbers of the insects caught by the light-diffusion board 2.

In an embodiment, the LEDs 32 gives off long-wavelength ultraviolet light (UV-A) in a wavelength range of 350-380 nm. In a preferred embodiment, the long-wavelength ultraviolet light is in a wavelength range of 355-370 nm to increase the attraction thereof to insects and thus improve insect catching rate.

The titanium dioxide photocatalyst 4 is arranged inside casing 1. The titanium dioxide photocatalyst 4 can be provided on metal by means of coating to contact the ultraviolet light emitting from the LEDs to generate an effect of sterilization and deodorization. Titanium dioxide, upon contact with ultraviolet light, generates hydroxide ions. Upon contact, hydroxide ions break down bacteria, germs, or decaying insect bodies that are suspended in the air to release water and carbon dioxide, so as to provide an effect of purification of air. In an embodiment, the titanium dioxide photocatalyst 4 is coated on a metallic mesh to provide an advantage of extended usage time life.

In an embodiment, the sanitizing and insect-trapping device 100 further comprises a fan 5. Fan 5 is disposed of inside casing 1 and is arranged to blow air and gases inside casing 1 to the outside of casing 1. Fan 5 may blow carbon dioxide resulting from the decomposition of insect bodies or germs out of casing 1. Since carbon dioxide attracts certain insects, such as mosquitos, the insect catch rate may be increased. On the other hand, fan 5 may also help draw external surrounding air into casing 1, so that by means of the ultraviolet light and the titanium dioxide photocatalyst 4, effects of circulation, sanitization, and purification of air may be achieved.

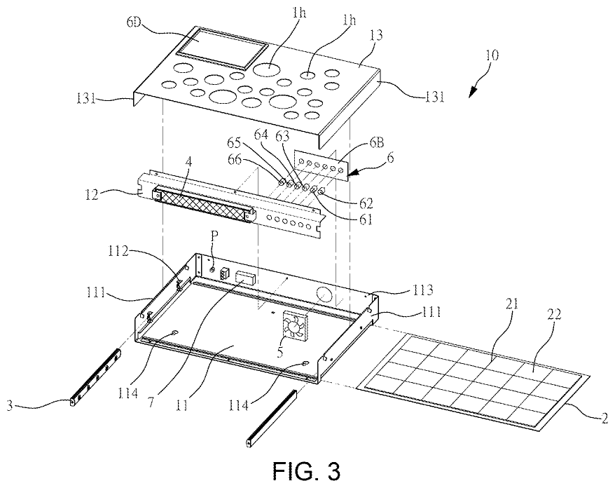

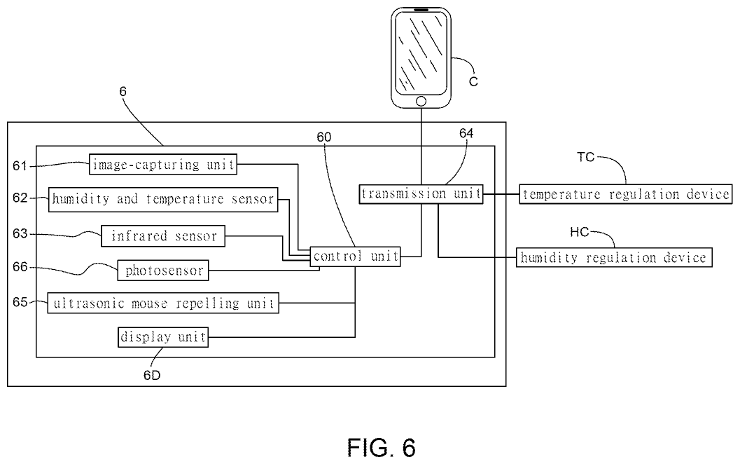

In an embodiment, casing 1 is further provided, in the interior thereof, with a monitoring module 6 to instantaneously analyze inside and outside environmental conditions of casing 1 and the types and numbers of insects caught thereby. As shown in FIGS. 3 and 6, in the instant embodiment, the monitoring module 6 comprises a control unit 60, an image-capturing unit 61, a humidity and temperature sensor 62, an infrared sensor 63, a transmission unit 64, an ultrasonic mouse repelling unit 65, and a photosensor 66. The image-capturing unit 61, the humidity and temperature sensor 62, the infrared sensor 63, the transmission unit 64, the ultrasonic mouse repelling unit 65, and the photosensor 66 are each in electrical connection with the control unit 60. The control unit 60 controls the operation of the image-capturing unit 61, the humidity and temperature sensor 62, the infrared sensor 63, the transmission unit 64, the ultrasonic mouse repelling unit 65, and the photosensor 66 and receives, stores, and analyze data transmitted back from the above-named components. The control unit 60 comprises a processor and a memory to edit, store, and execute program instructions and data. The control unit 60 can be embodied by means of a programmable and memory-included circuit or electronic chip.

The image-capturing unit 61 is arranged to capture images of insects caught by board 2 in order to analyze the types and numbers of the insects. In some embodiments, the control unit 60 is set in communication connection with a computation device C by means of the transmission unit 64 in order to transmit data of the images of the insects to the computation device C to allow an operator to proceed with the analysis of the types and numbers of the insects by means of the computation device C. The computation device C can be a personal computer, a workstation computer, a notebook computer, a mobile phone, or a personal digital assistant (PDA).

The humidity and temperature sensor 62 is arranged to detect the humidity and temperature of the surrounding environment and transmits signals indicative of humidity and temperature to the control unit 60 or the computation device C. Based on a relationship between the humidity and temperature data, the types and the numbers of the caught insects, and time (such as daytime, nighttime, or different seasons or months), the operator may properly handle and analyze the surrounding environmental condition of the site where the sanitizing and insect-trapping device install and may carry out data analysis to serve as a reference for insect control.

The infrared sensor 63 is in operation and collaboration with the control unit 60 and the ultrasonic mouse repelling unit 65 to detect and repel mice. The ultrasonic mouse repelling unit 65 is adjustable in respect of the frequency of the ultrasonic sound to prevent mice from immunization against fixed frequencies.

The photosensor 66 is arranged to detect the intensity of the ultraviolet light emitting from the LEDs 32 in order to identify and determine any deterioration of the ultraviolet light in respect of the intensity thereof and transmits signals to the control unit 60 or the computation device C to provide a reminder to a user to replace the LED light bar 3.

Further, in some embodiments, the control unit 60 is set in communication connection with a temperature regulation device TO and a humidity regulation device HO by means of the transmission unit 64. The operator may make direct adjustment of humidity and temperature on-site according to the relationship between the types and the numbers of the insects and the humidity and temperature data; alternatively, the operator may control the temperature regulation device TC and the humidity regulation device H from a remote site by means of the computation device C to adjust the humidity and temperature of the environment in order to reduce the appearance of certain specific insects in such a site.

The transmission unit 64 may perform, through for example wired or wireless measures (such as 3G, 4G, 5G, WiFi, Zigbee, or any other suitable communication technology) transmission of data and instructions between the control unit 60 and the computation device C, the temperature regulation device TC, and the humidity regulation device HC.

In one embodiment, the monitoring module 6 further comprises a display unit 6D. The display unit is provided on casing 1 and is in electrical connection with the control unit 60 to display date, time, temperature, humidity, images of insects caught on the insect trap board, and a message for reminding the operator to replace the light bar. Further, the display unit 6D may involve functions of integrating audio and video signals and displaying, so that the sanitizing and insect-trapping device 100 is made more useful and more fashionable. The display unit 6D can be a liquid crystal panel involving touch control.

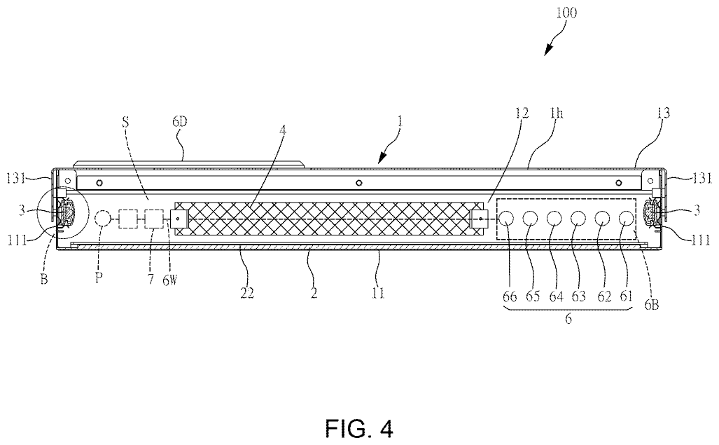

As shown in FIGS. 3 and 4, casing 1 is provided, in the interior thereof with a metal barrier plate 12. The metal barrier plate 12 defines, together with a wall 113 of casing 1, an installation space S. The circuit board 6B and the wirings 6 W of the monitoring module 6 are disposed of in the installation space S to prevent from being directly irradiated by ultraviolet light. This arrangement would extend the usage time life of the monitoring module 6.

In an embodiment, casing 1 comprises an electrical power unit 7. In some embodiments, the electrical power unit can be one of a direct-current power supply, an alternate-current power supply, a battery, a solar cell, or a combination thereof, which supplies electrical power for the LEDs 32, the monitoring module 6, the fan 5, and other components. The sanitizing and insect-trapping device 100 may also be equipped with an electrical power connection port P for connection with an external power supply for the electric power or charging the battery of the electrical power unit 7.

The present invention has a simplified and compact overall structure, a novel appearance, and high reliability and is easy to service and maintain and is easy to set up and operate in both indoor and outdoor conditions due to the overall size being small and lightweight, and may also be used as a decoration to improve the flexibility of utilization. The description of the embodiments provided above illustrates the use of the sanitizing and insect-trapping device of the present invention does not cause damage to human bodies due to the irradiation of ultraviolet light and is effective in air purification, sterilization, and deodorization and provides advantages of extended usage time life, energy-saving and environment protection, simplified and compact overall structure, and easy installation. When serving as an insect trap device, the present invention provides the convenience of instantaneous connection and operation with a remote control to help control insects. The sanitizing and insect-trapping device according to the present invention provides versatile utilization and protects the health and safety of human bodies.

What is claimed is:

1. A sanitizing and insect-trapping device, comprising:
a casing, the casing being formed with a plurality of openings;
at least one light-emitting diode (LED) light bar, the at least one LED light bar being arranged in an interior of the casing, each of the at least one LED light bar comprising a metal hood and a plurality of LEDs, the metal hood comprising a plurality of openings, wherein the LEDs is disposed inside the metal hood at a location corresponding to the openings of the metal hood and is configured to emit ultraviolet light;
a board, which comprises a light diffusion agent, the board is arranged in the interior of the casing and being exposed to the ultraviolet light emitting from the LEDs; and
titanium dioxide photocatalyst, which is arranged in the interior of the casing, wherein the titanium dioxide photocatalyst is disposed of on metal and is exposed to the ultraviolet light emitting from the LEDs.

2. The sanitizing and insect-trapping device according to claim 1, wherein the LEDs have a height that is lower than the height of the openings of the metal hood.

3. The sanitizing and insect-trapping device according to claim 2, further comprising a fan, which is disposed of in the casing, the fan is arranged to draw external air into the casing and to blow air and gases inside the casing to outside of the casing so as to circulate air.

4. The sanitizing and insect-trapping device according to claim 1, wherein the LEDs are arranged to emit long-wavelength ultraviolet light, the long-wavelength ultraviolet light having a wavelength in the range of 350-380 nm.

5. The sanitizing and insect-trapping device according to claim 4, wherein the wavelength of the long-wavelength ultraviolet light is in the range of 355-370 nm.

6. The sanitizing and insect-trapping device according to claim 1, wherein the board has a surface that is coated with insect sticking, glue.

7. The sanitizing and insect-trapping device according to claim 6, wherein the casing is further provided, in the interior thereof, with a monitoring module and a metal barrier plate, wherein the metal barrier plate defines, together with a wall of the casing, an installation space, and the monitoring module and a circuit board and wirings of the monitoring module are disposed of in the installation space.

8. The sanitizing and insect-trapping device according to claim 7, wherein the monitoring module comprises a control unit, an image-capturing unit, a humidity and temperature sensor, an infrared sensor, a transmission unit, an ultrasonic mouse repelling unit, and a photosensor, and the image-capturing unit, the humidity and temperature sensor, the infrared sensor, the transmission unit, the ultrasonic mouse repelling unit, and the photosensor are each in electrical connection with the control unit.

9. The sanitizing and insect-trapping device according to claim 8, wherein the monitoring module further comprises a display unit, and the display unit is disposed of on the casing and in electrical connection with the control unit.

10. The sanitizing and insect-trapping device according to claim 8, wherein the control unit is in communication connection with a temperature regulation device and a humidity regulation device by means of the transmission unit.

11. The sanitizing and insect-trapping device according to claim 10, wherein the control unit is in communication connection with a computation device by means of the transmission unit.

12. The sanitizing and insect-trapping device according to claim 1, wherein the casing is provided with an electrical power unit to supply electrical power.

13. The sanitizing and insect-trapping device according to claim 12, wherein the electrical power unit comprises one of a direct-current power supply, an alternate-current power supply, a battery, and a solar cell, or a combination thereof.

14. The sanitizing and insect-trapping device according to claim 1, wherein the casing is provided with a wall-mounting aperture for mounting to a wall.

15. The sanitizing and insect-trapping device according to claim 6, wherein the board has a surface that is formed with grid lines.

16. The sanitizing and insect-trapping device according to claim 1, wherein the casing is made of an anti-ultraviolet material and the at least one LED light bar is fixed in the interior of the casing by means of at least one metal clip.
